(19) United States
(12) Patent Application Publication
(10) Pub. No.: US 2025/0339644 A1
(43) Pub. Date: Nov. 6, 2025

(54) DIRECTIONALITY INDICATION FOR MEDICAL INSTRUMENT DRIVING

(71) Applicant: Auris Health, Inc., Santa Clara, CA (US)

(72) Inventors: Daniel Lau, Oakland, CA (US); Chiara Gatti, San Francisco, CA (US); Sean Taylor Fielding, Belmont, CA (US); Jacob William Caldwell, San Francisco, CA (US)

(73) Assignee: Auris Health, Inc., Santa Clara, CA (US)

(21) Appl. No.: 19/190,176

(22) Filed: Apr. 25, 2025

Related U.S. Application Data
(60) Provisional application No. 63/641,920, filed on May 2, 2024.

(51) Int. Cl. A61M 25/01; A61B 1/00; A61B 1/008; A61B 1/05

(52) U.S. Cl. CPC A61M 25/0105; A61B 1/00039; A61B 1/008; A61B 1/05; A61M 2025/0166

(57) ABSTRACT

This disclosure provides methods, devices, and systems for manipulating a medical instrument from the perspective of another medical instrument (such as an endoscope). The present implementations more specifically relate to techniques for controlling a medical instrument having an elongate shaft that is symmetrical in shape about its roll axis (such as a catheter). When a catheter is within a field-of-view (FOV) of a camera disposed on an endoscope, the catheter can be seen in real-time images captured by the camera. In some aspects, the elongate shaft may include markings or labels on its outer surface so that it appears asymmetric about its roll axis. Example suitable markings can include any visual cue or other indication that reduces or eliminates the rotational symmetry of the catheter (such as colors, shapes, lights, patterns, and/or various other features that can be seen in the FOV of the camera or projected onto the images).

### CROSS-REFERENCE TO RELATED APPLICATIONS

This application claims priority and benefit under 35 U.S.C. § 119 (e) to U.S. Provisional Patent Application No. 63/641,920, filed May 2, 2024, which is incorporated herein by reference in its entirety.

### TECHNICAL FIELD

This disclosure relates generally to medical systems, and specifically to directionality indication techniques for medical instrument driving.

### DESCRIPTION OF RELATED ART

Many medical procedures, such as laparoscopy, ureteroscopy, or percutaneous nephrolithotomy (PCNL), involve a series of complex steps that require careful movement and positioning of medical tools or instruments inside and/or outside a patient's body. For example, to remove urinary stones from the bladder and ureter, a medical provider (such as a physician or a technician) can insert a ureteroscope into the urinary tract through the urethra. A ureteroscope includes an endoscope at its distal end configured to enable visualization of the urinary tract. During some percutaneous access procedures, the ureteroscope can be used to designate or set a target location for a needle to access the kidney percutaneously. The medical provider inserts the needle into the patient, to the target location, and proceeds to dilate the tract and perform a PCNL procedure. For example, the medical provider may use another medical instrument (which may be in conjunction with the needle) to extract the stone from the kidney via the percutaneous access point. The success or failure of a percutaneous access procedure often depends on various factors, including the medical provider's skill, the patient's anatomy, as well as the quality and design of any tools and/or equipment the medical provider uses to perform the procedure.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a medical instrument. The medical instrument includes an elongate shaft having a distal end configured to articulate in a plurality of directions orthogonal to its roll axis; a first visual feature disposed on a first portion of the elongate shaft associated with a first direction of the plurality of directions; and a second visual feature disposed on a second portion of the elongate shaft different than the first portion so that the elongate shaft is visually asymmetric about the roll axis.

Another innovative aspect of the subject matter of this disclosure can be implemented in a system including a medical instrument having an elongate shaft and a robotic arm configured to manipulate the medical instrument. The system further includes control circuitry configured to receive user input for articulating a distal end of the elongate shaft in a plurality of directions orthogonal to its roll axis; control the robotic arm to manipulate the medical instrument based on the received user inputs; and display a graphical interface including an image depicting the medical instrument in a field-of-view (FOV) of a camera and a visual indicator indicating a mapping of the plurality of directions to the medical instrument in the FOV of the camera.

Another innovative aspect of the subject matter of this disclosure can be implemented in a method for controlling a medical instrument having an elongate shaft. The method includes steps of receiving user input for articulating a distal end of the elongate shaft in a plurality of directions orthogonal to its roll axis; controlling a robotic arm to manipulate the elongate shaft based on the received user inputs; and displaying a graphical interface including an image depicting the elongate shaft in an FOV of a camera and a visual indicator indicating a mapping of the plurality of directions to the elongate shaft in the FOV of the camera.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows an example of the robotic system depicted in FIG. 1, according to some implementations.

FIG. 3 shows an example of the control system depicted in FIG. 1, according to some implementations.

FIGS. 4A and 4B show an example controller for a robotic system, according to some implementations.

FIGS. 5A-5C show an example medical instrument, according to some implementations.

FIGS. 6A and 6B show another example medical instrument, according to some implementations.

FIG. 7 shows another example medical instrument, according to some implementations.

FIG. 8 shows an example graphical interface for controlling a medical instrument, according to some implementations.

FIG. 9A shows another example graphical interface for controlling a medical instrument, according to some implementations.

FIG. 9B shows another example graphical interface for controlling a medical instrument, according to some implementations.

FIG. 10 shows a block diagram of an example system for controlling a medical instrument, according to some implementations.

FIG. 11 shows an illustrative flowchart depicting an example operation for controlling a medical instrument having an elongate shaft, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures involve a series of complex steps that require careful movement and positioning of medical tools or instruments inside a patient's body. Some medical procedures can now be performed, at least in part, by a robotic system or apparatus, which can aid the physician in navigating or positioning such medical instruments. For example, to perform a robotically-assisted percutaneous nephrolithotomy (PCNL) procedure, a physician controls a robotic system to advance and navigate a medical instrument (such as a scope) from the urethra, through the bladder, up the ureter, and into the kidney where the kidney stone is located. The robotic system may include, or may be coupled to, one or more display devices that can provide information to assist the physician in navigating the medical instrument. Example suitable information can include real-time images captured by the medical instrument, a visualization to indicate a position and/or orientation (also referred to as a “pose”) of the medical instrument, and/or guidance regarding the procedure, among other examples. Such information can be captured or obtained using various sensors and/or cameras disposed on or proximate to the medical instrument.

Some medical procedures involve the use of shaft-type instruments, such as catheters, which can be inserted percutaneously into an anatomy. Catheters are often driven from the perspective or point-of-view of another medical instrument inserted in the anatomy (such as an endoscope). For example, when a catheter is within a field-of-view (FOV) of a camera disposed on an endoscope, the catheter can be seen in real-time images captured by the camera and displayed on an interface of the robotic system. However, many existing catheters are cylindrical in shape and rotationally symmetric about the roll axis. As a result, a user may be unable to distinguish the catheter's pitch axis from its yaw axis in the real-time images, which can complicate driving of the catheter from the perspective of the endoscope. Aspects of the present disclosure recognize that the orientation of the catheter can be more readily discerned by marking or labeling one or more sides of the catheter so that it appears asymmetric about its roll axis. Example suitable markings can include any visual cue or other indication that reduces or eliminates the rotational symmetry of the catheter (such as colors, shapes, lights, patterns, and/or various other features that can be seen in the FOV of the camera or projected onto the images).

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled, at least in part, by a human operator (such as a physician or a technician). In some other implementations, the robotic tools may operate in an autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. However, as mentioned, description of the renal or urinary anatomy and associated medical issues and procedures is presented herein to aid in the description of the concepts disclosed herein. In some implementations, the techniques and systems described herein are discussed in the context of a percutaneous procedure, which can include any procedure where access is gained to a target location by making a puncture or incision in the skin, mucous membrane, or other body layer. However, it should be understood that these techniques and systems can be implemented in the context of any medical procedure.

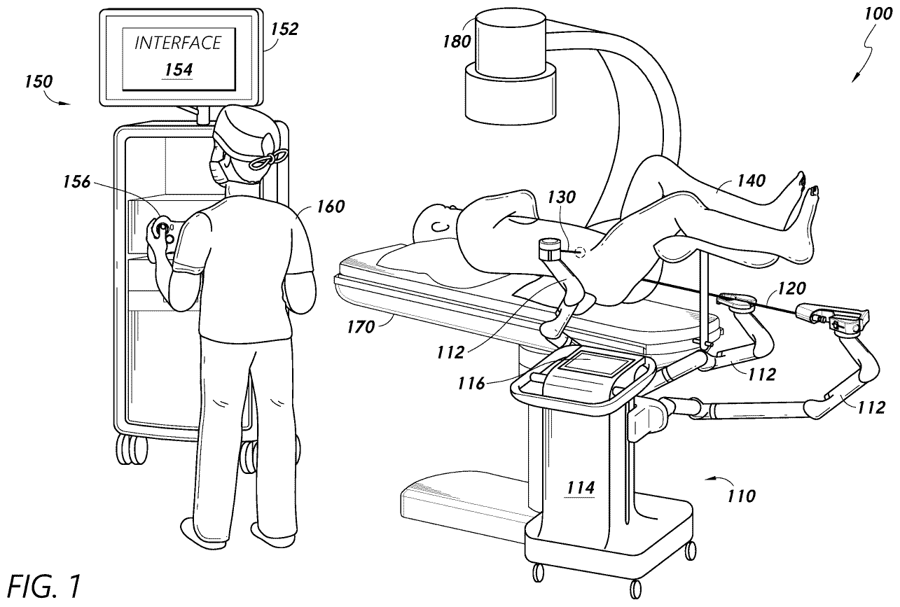

FIG. 1 shows an example medical system 100, according to some implementations. The medical system 100 includes a robotic system 110 configured to engage with and/or control a medical instrument 120, a medical instrument 130, and/or another medical instrument to perform a procedure on a patient 140. The medical system 100 also includes a control system 150 configured to interface with the robotic system 110, provide information regarding the procedure, and/or perform a variety of other operations. For example, the control system 150 can include a display(s) 152 to present certain information to assist a physician 160. The medical system 100 can include a table 170 configured to hold the patient 140. In some implementations, the medical system 100 can also include an imaging device 180 which can be integrated into a C-arm and/or configured to provide imaging during a procedure, such as for a fluoroscopy-type procedure. Various acts are described herein as being performed by the physician 160. It should be understood that these acts can be performed directly by the physician 160, a user under the direction of the physician 160, another user (such as a technician), a combination thereof, and/or any other user.

In the example of FIG. 1, the medical instrument 120 is implemented as a scope and the medical instrument 130 is implemented as a catheter. Thus, for ease of discussion, the medical instrument 120 is referred to as “the scope 120” or “the direct access/entry instrument 120,” and the medical instrument 130 is referred to as “the catheter 130” or “the percutaneous access/entry instrument 130.” However, the medical instrument 120 and the medical instrument 130 can each be implemented as any type of medical instrument including, for example, a scope (sometimes referred to as an “endoscope”), a catheter, a needle, a guidewire, a lithotripter, a basket retrieval device, forceps, a vacuum, a scalpel, an imaging probe, jaws, scissors, graspers, needle holder, micro dissector, staple applier, tacker, suction/irrigation tool, clip applier, and so on. In some implementations, a medical instrument is a steerable device, while in other implementations a medical instrument is a non-steerable device. In some implementations, a surgical tool refers to a device that is configured to puncture or to be inserted through the human anatomy, such as a needle, a scalpel, a guidewire, and so on. However, a surgical tool can refer to other types of medical instruments.

The term “scope” or “endoscope” are used herein according to their broad and ordinary meanings and can refer to any type of elongate medical instrument having image generating, viewing, and/or capturing functionality and configured to be introduced into any type of organ, cavity, lumen, chamber, and/or space of a body. For example, references herein to scopes or endoscopes can refer to a ureteroscope (such as for accessing the urinary tract), a laparoscope, a nephroscope (such as for accessing the kidneys), a bronchoscope (such as for accessing an airway, such as the bronchus), a colonoscope (such as for accessing the colon), an arthroscope (such as for accessing a joint), a cystoscope (such as for accessing the bladder), a borescope, and so on. Scopes/endoscopes, in some instances, may comprise a rigid or flexible tube, and may be dimensioned to be passed within an outer sheath, catheter, introducer, or other lumen-type device, or may be used without such devices.

The terms “direct entry” or “direct access” are used herein according to their broad and ordinary meaning and may refer to any entry of instrumentation through a natural or artificial opening in a patient's body. For example, with reference to FIG. 1, and as noted above, the scope 120 may be referred to as a direct access instrument, since the scope 120 enters into the urinary tract of the patient 140 via the urethra.

The terms “percutaneous entry” or “percutaneous access” are used herein according to their broad and ordinary meaning and may refer to entry, such as by puncture and/or minor incision, of instrumentation through the skin of a patient and any other body layers necessary to reach a target anatomical location associated with a procedure (such as the calyx network of the kidney). As such, a percutaneous access instrument may refer to a medical instrument, device, or assembly that is configured to puncture or to be inserted through skin and/or other tissue/anatomy, such as a needle, scalpel, guidewire, sheath, shaft, scope, catheter, and the like. However, it should be understood that a percutaneous access instrument can refer to other types of medical instruments in the context of the present disclosure. In some implementations, a percutaneous access instrument refers to an instrument/device that is inserted or implemented with a device that facilitates a puncture and/or minor incision through the skin of a patient. For example, the catheter 130 may be referred to as a percutaneous access instrument when the catheter 130 is inserted through a sheath/shaft that has punctured the skin of the patient 140.

In some implementations, a medical instrument, such as the scope 120 and/or the catheter 130, includes a sensor (sometimes referred to as a position sensor) that is configured to generate sensor data. In examples, sensor data can indicate a position and/or orientation of the medical instrument and/or can be used to determine a position and/or orientation of the medical instrument. For instance, sensor data can indicate a position and/or orientation of a scope, which can include a roll of a distal end of the scope. A position and orientation of a medical instrument can be referred to as a pose of the medical instrument. A sensor can be positioned on a distal end of a medical instrument and/or any other location. In some implementations, a sensor can provide sensor data to the control system 150 and/or another system/device to perform one or more localization techniques to determine/track a position and/or an orientation of a medical instrument.

In some implementations, a sensor can include an electromagnetic (EM) sensor with a coil of conductive material. Here, an EM field generator can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a sensor can include another type of sensor, such as a camera, a range sensor, a radar device, a shape sensing fiber, an accelerometer, a gyroscope, an accelerometer, a satellite-based positioning sensor (such as a global positioning system (GPS)), a radio-frequency transceiver, and so on.

A medical instrument can be associated with a coordinate frame, which can include a set of two or more vectors (or axes) that make a right angle with one another. For example, in a three-dimensional space, a coordinate frame can include three vectors (such as x-vector, y-vector, and z-vector) that make right angles with each other. Although various conventions can be used, for ease of illustration the description herein will often refer to the “forward” direction (such as insert/retract) as corresponding to positive z, the “right” direction as corresponding to positive x, and the “up” direction as corresponding to positive y. The z-vector can extend along a longitudinal axis of a medical instrument. Such coordinate system can be referred to as a “left-handed coordinate system.” However, the disclosure herein can similarly be discussed/implemented in the context of a right-handed coordinate system. In examples, a coordinate frame is set/correlated based on a position of one or more elongate movement members of a medical device (such as one or more pull wires). Further, in examples, a coordinate frame is set/correlated to/based on a position of an image device on a medical instrument, such as a distal end of an image device on a tip of a scope. As such, a coordinate frame may correspond to a camera frame of reference. However, a coordinate frame can be correlated/set at other locations. In many examples, a coordinate frame for a medical instrument will be represented/discussed with respect to a distal end of a medical instrument (such as an end at a treatment site). However, a coordinate frame can be positioned elsewhere.

As noted above, the control system 150 can be configured to provide various functionality to assist in performing a medical procedure. In some implementations, the control system 150 can be coupled to the robotic system 110 and operate in cooperation with the robotic system 110 to perform a medical procedure on the patient 140. For example, the control system 150 can communicate with the robotic system 110 via a wireless or wired connection to control the scope 120 and/or the catheter 130 connected to the robotic system 110, receive an image(s) captured by the scope 120, and so on. Additionally, or alternatively, the control system 150 can provide fluids to the robotic system 110 via one or more fluid channels, provide power to the robotic system 110 via one or more electrical connections, provide optics to the robotic system 110 via one or more optical fibers or other components, and so on. In some implementations, the control system 150 can communicate with the scope 120 (and/or the catheter 130) to receive sensor data (via the robotic system 110 and/or directly from the scope 120 and/or the catheter 130). In examples, sensor data can indicate or be used to determine a position and/or orientation of a medical instrument. Moreover, in some implementations, the control system 150 can communicate with the table 170 to position the table 170 in a particular orientation or otherwise control the table 170. Further, in some implementations, the control system 150 can communicate with an EM field generator to control generation of an EM field around the patient 140.

The control system 150 includes various I/O devices configured to assist the physician 160 or others in performing a medical procedure. In the example of FIG. 1, the control system 150 includes an I/O device(s) 156 that is employed by the physician 160 or other user to navigate or otherwise control a medical instrument. For example, the physician 160 can provide input via the I/O device(s) 156 and, in response, the control system 150 can send control signals to the robotic system 110 to manipulate the scope 120 and/or the catheter 130. In examples, the physician 160 can use the same I/O device to control the scope 120 and/or the catheter 130 (such as switch control between the devices). In some implementations, the scope 120 is driven from a first-person perspective (such as from the viewpoint of the scope 120) and/or the catheter 130 is driven from a third-person perspective (such as from the viewpoint of the scope 120), as discussed in further detail below. Although the I/O device(s) 156 is illustrated as a controller in the example of FIG. 1, the I/O device(s) 156 can be implemented as a variety of types of I/O devices, such as a touchscreen, a touch pad, a mouse, a keyboard, etc.

As also shown in FIG. 1, the control system 150 can include a display(s) 152 to provide various information related to a procedure. For example, the control system 150 can receive real-time images that are captured by the scope 120 and display the real-time images and/or visual representations of the real-time images via the display(s) 152. The display(s) 152 can present an interface(s) 154, such as any of the interfaces discussed herein, which can include image data from the scope 120 and/or another medical instrument.

In some implementations, the control system 150 can provide image data via the interface(s) 154 in a manner that maintains a constant orientation of the image data (sometimes referred to as an “original image view”). For example, the interface(s) 154 can maintain a constant relationship with a coordinate frame for the scope 120 (so that up in the interface(s) 154 corresponds to the positive y-vector of the coordinate frame for the scope 120). To illustrate, assume that a kidney stone depicted in image data from the scope 120 initially shows up on the left side in the interface(s) 154. If the scope rolls 180 degrees, the kidney stone will move within the interface(s) 154 during the roll and appear on the right side in the interface(s) 154 after the roll. Here, the control system will not adjust the orientation of the image data displayed through the interface(s) 154. As such, the horizon in the image data can be perceived as rolling.

In other implementations, the control system 150 can provide image data via the interface(s) 154 in a manner that updates an orientation of the image data (sometimes referred to as a “rotated image or virtual view”). For example, the interface(s) 154 can update a relationship with a coordinate frame for the scope 120 (so that up in the interface(s) 154 does not always correspond to the positive y-vector of the coordinate frame for the scope 120). To illustrate, assume that a kidney stone depicted in image data from the scope 120 initially shows up on the left side in the interface(s) 154. If the scope rolls 180 degrees, the kidney stone will still show up on the left side in the interface(s) 154 after the roll. Here, the control system 150 can adjust the orientation of the image data displayed via the interface(s) 154 as the scope 120 rolls 180 degrees to maintain objects depicted in the image data in the same orientation (such as to roll correct the image data). As such, the horizon in the image data can be perceived as staying the same.

Additionally, or alternatively, the control system 150 can output other information via the display(s) 152. For example, the control system 150 can receive signals (such as analog, digital, electrical, acoustic/sonic, pneumatic, tactile, hydraulic, etc.) from a medical monitor and/or a sensor associated with the patient 140, and the display(s) 152 can present information regarding the health or environment of the patient 140. Such information can include information that is displayed via a medical monitor including, for example, a heart rate (such as ECG, HRV, etc.), blood pressure/rate, muscle bio-signals (such as EMG), body temperature, blood oxygen saturation (such as Sp(h), CO2, brainwaves (such as EEG), environmental and/or local or core body temperature, and so on.

To facilitate the functionality of the control system 150, the control system 150 can include various components (sometimes referred to as “subsystems”). For example, the control system 150 can include control electronics/circuitry, as well as one or more power sources, pneumatics, optical sources, actuators, memory/data storage devices, and/or communication interfaces. In some implementations, the control system 150 includes control circuitry comprising a computer-based control system that is configured to store executable instructions, that when executed, cause various operations to be implemented. In some implementations, the control system 150 may be movable (such as shown in FIG. 1). In some other implementations, the control system 150 may be a stationary system. Although various functionality and components are discussed as being implemented by the control system 150, any of this functionality and/or components can be integrated into and/or performed by other systems and/or devices, such as the robotic system 110, the table 170, or even the scope 120 and/or the catheter 130.

The robotic system 110 can be arranged in a variety of ways depending on the particular procedure. The robotic system 110 can include the one or more robotic arms 112 configured to engage with and/or control a medical instrument(s) to perform a procedure. As shown, each robotic arm 112 can include multiple arm segments coupled to joints, which can provide multiple degrees of movement. In the example of FIG. 1, two of the robotic arms 112 are actuated to engage with the scope 120 to access a target site through the urethra of the patient 140, and one of the robotic arms 112 is actuated to engage with the catheter 130 to access the target site through a percutaneous access path. When the robotic system 110 is properly positioned, the scope 120 and/or the catheter 130 can be inserted and/or navigated into the patient 140 robotically using the robotic arms 112, manually by the physician 160, or a combination thereof. Although not illustrated in FIG. 1, the robotic arms 112 can also be connected to other medical instruments, which may be interchanged during a procedure, such as an electromagnetic (EM) field generator that may be positioned near a treatment site during a particular phase of a procedure. Further, although the robotic arms 112 are shown in various positions and coupled to various instrumentation, it should be understood that such configurations are shown for convenience and illustration purposes, and such robotic arms 112 may have different configurations over time during a medical procedure.

The robotic system 110 can also include a support structure 114 coupled to the one or more robotic arms 112. The support structure 114 can include control electronics/circuitry, one or more power sources, one or more pneumatics, one or more optical sources, one or more actuators (such as motors to move the one or more robotic arms 112), memory/data storage, and/or one or more communication interfaces. In some implementations, the support structure 114 includes an input/output (I/O) device(s) 116 configured to receive input, such as user input to control the robotic system 110, and/or provide output, such as a graphical user interface (GUI), information regarding the robotic system 110, information regarding a procedure, and so on. The I/O device(s) 116 can include a display, a touchscreen, a touchpad, a projector, a mouse, a keyboard, a microphone, a speaker, etc. In some implementations, the robotic system 110 is movable (such as the support structure 114 includes wheels) so that the robotic system 110 can be positioned in a location that is appropriate or desired for a procedure. In other implementations, the robotic system 110 is a stationary system. Further, in some implementations, the robotic system 112 is integrated into the table 170.

The robotic system 110 can be coupled to any component of the medical system 100, such as the control system 150, the table 170, the scope 120, the catheter 130, and/or other devices/instruments. In one example, the robotic system 110 is communicatively coupled to the control system 150 to receive a control signal from the control system 150 to perform an operation, such as to position a robotic arm 112 in a particular manner, manipulate the scope 120 and/or the catheter 130, and so on. In another example, the robotic system 110 is configured to receive an image from the scope 120 depicting internal anatomy of the patient 140 and/or send the image to the control system 150, which can then be displayed on the display(s) 152. Furthermore, in some implementations, the robotic system 110 is coupled to a component of the medical system 100, such as the control system 150, in such a manner as to allow for fluids, optics, power, or the like to be received therefrom.

The imaging device 180 can be configured to capture/generate one or more images of the patient 140 during a procedure, such as one or more x-ray or CT images. In examples, images from the imaging device 180 can be provided in real-time to view anatomy and/or medical instruments, such as the scope 120 and/or the catheter 130, within the patient 140 to assist the physician 160 in performing a procedure. The imaging device 180 can be used to perform a fluoroscopy (such as with a contrast dye within the patient 140) or another type of imaging technique. Although shown in FIG. 1, in implementations the imaging device 180 is not implemented for performing a procedure and/or the imaging device 180 (including the C-arm) is eliminated.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, etc. Further, in some implementations, the components of the medical system 100 are connected for data communication, fluid/gas exchange, power exchange, and so on, via one or more support cables, tubes, or the like.

As noted above, the medical system 100 can enable the physician 160 to drive a medical instrument, such as to navigate the medical instrument within the patient 140. For example, the control system 150 can receive an input signal from the I/O device(s) 156 indicative of a direction of movement for a medical instrument. The control system 150 can determine an orientation/position of the medical instrument, an orientation/position of another medical instrument providing image data of the medical instrument (in some cases), and/or an orientation of image data displayed through the interface(s) 154. The control system 150 can use such information to generate a control signal to move the medical instrument in the appropriate direction relative to a coordinate/control frame of the medical instrument. The control system 150 can send the control signal to the robotic system 110 to manipulate the medical instrument.

In some implementations, the medical system 100 enables the physician 160 to drive a medical instrument from a perspective of the medical instrument (also referred to as “first-person driving”). This type of driving may be useful in situations where a medical instrument includes an imaging device to provide image data and/or in other situations. For example, the control system 150 can enable the scope 120 to be driven from a perspective of the scope 120. The scope 120 can include an imaging device configured to provide image data to the control system 150. The control system 150 can display the image data through the interface(s) 154 to assist the physician 160 in driving the scope 120 from the perspective of the scope 120.

In the case of first-person driving, the control system 150 can generally control a medical instrument to move in a correlated manner to an orientation of image data from the medical instrument displayed via the interface(s) 154. For example, assume that the physician 160 is driving the scope 120 from the perspective of the scope 120 and the physician 160 provides input via the I/O device(s) 156 to move the scope 120 in an upward direction relative to the I/O device(s) 156, such as by selecting an up control on the I/O device(s) 156. The control system 150 can determine an orientation/position of the scope 120 and/or an orientation of image data from the scope 120 that is displayed via the interface(s) 154. The control system 150 can use such orientation/position information to move the scope 120 in the appropriate direction that shows up as an upward direction on the interface(s) 154.

To illustrate, if the interface(s) 154 is displaying a static view for the scope 120 (such as where up in the interface(s) 154 corresponds to the positive y-vector of the coordinate frame for the scope 120), the control system 150 can cause the scope 120 to move along the positive y-vector of the coordinate frame for the scope 120 in response to up input on the I/O device(s) 156. Further, if the interface(s) 154 is displaying a rotated image view for the scope 120 (such as up in the interface(s) 154 does not always correspond to the positive y-vector of the coordinate frame for the scope 120), the control system 150 can determine an offset of a frame of reference of the image data to the coordinate frame for the scope 120 to cause the scope 120 to move in the appropriate direction that appears as the scope 120 moving upward in the interface(s) 154.

Further, in some implementations, the medical system 100 can enable the physician 160 to drive a medical instrument from a perspective of another medical instrument (also referred to as “third-person driving”). This type of driving may be useful in situations where medical instruments rendezvous with each other and/or one of the medical instruments does not include an imaging device. For example, the control system 150 can enable the physician 160 to drive the catheter 130 from the perspective of the scope 120, which may be useful in cases where the catheter 130 does not include an imaging device. Here, the scope 120 can provide image data and the control system 150 can display the image data through the interface(s) 154. When the catheter 130 is within a field-of-view (FOV) of the scope 120, the physician 160 can view the catheter 130 in the interface(s) 154 and drive the catheter 130 from the perspective of the scope 120.

In the case of third-person driving, the control system 150 can generally implement a control scheme to control movement of a medical instrument. For example, assume that the physician 160 is driving the catheter 130 from the perspective of the scope 120 and the physician 160 provides input via the I/O device(s) 156 to move the catheter 130 in an upward direction relative to the I/O device(s) 156, such as by selecting an up control on the I/O device(s) 156. The control system 150 can implement a control scheme for the catheter 130 that accounts for an orientation of the scope 120 relative to the catheter 130. This can enable the catheter 130 to move in the appropriate direction that shows up as an upward direction on the interface(s) 154.

A control scheme can be used to map inputs to control signals to move a medical instrument. In some implementations, a control scheme includes a control frame (sometimes referred to as a “control frame of reference”), which can include an abstract coordinate frame/set of vectors that is used to control a medical instrument/device. For example, a control frame can include a set of two or more vectors (or axes) that make right angles with one another. A control frame can generally be correlated to a coordinate frame for a medical instrument. For example, a control frame for a medical instrument can be offset with respect to a coordinate frame for the medical instrument (such as 30-degree offset about an axis/vector). In examples, a coordinate frame remains static for a medical instrument (i.e., fixed to a point on the medical instrument), while a control frame can be dynamically updated, such as based on roll of the medical instrument, an orientation of image data via a user interface, and the like. In examples, a control frame is correlated to a tip of a medical instrument. However, a control frame can be correlated/centered at other locations.

In some situations of third-person driving, it may be challenging for the physician 160 to drive a medical instrument. For example, if the physician 160 is driving the catheter 130 from the perspective of the scope 120 and the catheter 130 is facing the scope 120 in a substantially head on manner (such that a tip of the catheter 130 is facing a tip of the scope 120), the physician 160 may provide inverted left and right input to move the catheter 130 in the appropriate direction relative to the interface(s) 154. For instance, if the physician 160 desires to move the catheter 130 to the left with respect to the interface(s) 154, the physician 160 may provide right input via the I/O device(s) 156, and vice versa. In contrast, when the catheter 130 and the scope 120 are facing in substantially the same direction (such that a tip of the catheter 130 and a tip of the scope 120 are facing the same direction), no such inverted input is required.

As such, the medical system 100 can facilitate one or more control/driving modes to assist the physician 160 in driving a medical instrument. By using multiple control modes, a medical instrument can be driven in an effective manner for different orientations of the medical instruments relative to each other. For example, if the catheter 130 is being driven from the perspective of the scope 120, the physician 160 may be able to view the catheter 130 as moving in a direction on the interface(s) 154 that more intuitively corresponds to input provided via the I/O device(s) 156. In some examples, the medical system 100 can switch to a different control mode by reconfiguring the control system 150 (such as to process an input signal from the I/O device(s) 156 and/or to generate a control signal for the robotic system 110 in a different manner), reconfiguring the I/O device(s) 156 (such as to send a different input control signal), and/or reconfiguring the robotic system 110 (such as to control a robotic arm in a different manner). Although multiple control modes are often discussed in the context of third-person driving, such control modes can be used in other contexts, such as first-person driving or any other driving scenario.

In some implementations, the control system 150 can implement a direct control mode (also referred to as a “parallel mode”) to drive a medical instrument in a corresponding manner with respect to a coordinate/control frame of the medical instrument. For example, when driving the catheter 130 from the perspective of the scope 120 in the direct control mode, if the physician 160 selects left input on the I/O device(s) 156, the control system 150 can control the catheter 130 to move left with respect to the catheter 130. If the catheter 130 is facing in substantially the same direction as the scope 120, the physician 160 may view the catheter 130 as moving to the left in the interface(s) 154 (such as from the third-person point-of-view). In contrast, if the catheter 130 is facing the scope 120 in a head on manner, the physician 160 may view the catheter 130 as moving to the right in the interface(s) 154. Thus, the direct control mode may be implemented when the catheter 130 and the scope 120 are substantially facing in the same direction.

Additionally, or alternatively, the control system 150 can implement an inverted control mode (also referred to as a “mirrored mode”) to drive a medical instrument in an inverted manner with respect to a coordinate/control frame of the medical instrument. For example, when driving the catheter 130 from the perspective of the scope 120 in the inverted control mode, if the physician 160 selects left input on the I/O device(s) 156, the control system 150 can control the catheter 130 to move right with respect to the catheter 130. If the catheter 130 is facing the scope 120 in a head on manner, the physician 160 may view the catheter 130 as moving to the left in the interface(s) 154 (such as from the third-person point-of-view). In contrast, if the catheter 130 is facing in substantially the same direction as the scope 120, the physician 160 may view the catheter 130 as moving to the right in the interface(s) 154. Thus, the direct control mode may be implemented when the catheter 130 and the scope 120 are substantially facing each other in a head on manner.

In some implementations, the inverted control mode is associated with inverting horizontal movement for a medical instrument and not vertical movement. For example, in the inverted control mode, the control system 150 can cause a horizontal component for a coordinate/control frame to be inverted and not a vertical component(s) (such as invert a sign of an x value and not a y value). However, vertical directions can also be inverted in some cases of the inverted control mode.

Further, in some implementations, a control mode can indicate whether to invert retraction and insertion. For example, in the direct control mode, a forward input control on the I/O device(s) 156 can be associated with inserting the catheter 130 farther into the patient 140 and a reverse input control on the I/O device(s) 156 can be associated with retracting the catheter 130 from the patient 140. If the catheter 130 is being driven from the perspective of the scope 120 and the instruments are facing each other in a head on manner, the physician 160 may view the catheter 130 as moving closer to the scope 120 for forward input and as moving away from the scope 120 for reverse input. Further, in the inverted control mode, the forward input control can be associated with retracting the catheter 130 and the reverse input control can be associated with inserting the catheter 130. If the catheter 130 is being driven from the perspective of the scope 120 and the instruments are facing each other in a head on manner, the physician 160 may view the catheter 130 as moving away from the scope 120 for forward input and as moving closer to the scope 120 for reverse input.

In one illustrative percutaneous procedure, the medical system 100 can be used to remove a kidney stone from the patient 140 through a percutaneous access path. For example, the physician 160 can interact with the control system 150 (such as via an I/O device(s) 156) to cause the robotic system 110 to advance and/or navigate the scope 120 from the urethra, through the bladder, up the ureter, and into the kidney where the stone is located. The control system 150 can provide information via the display(s) 152 regarding the scope 120 to assist the physician 160 in navigating the scope 120, such as real-time images captured therewith. In examples, the scope 120 can be driven from a first-person perspective (such as from the viewpoint of the scope 120). Once at the site of the kidney stone (such as within a calyx of the kidney), the scope 120 can be used to designate/tag a target location for the catheter 130 to access the kidney percutaneously. To minimize damage to the kidney and/or the surrounding anatomy, the physician 160 can designate a particular papilla as the target location for entering into the kidney percutaneously with the catheter 130. However, other target locations can be designated or determined.

The physician 160 can also interact with the control system 150 to cause the robotic system 110 to advance and/or navigate the catheter 130 through a percutaneous access path to the target location designated by the scope 120. In some implementations, a needle or another medical instrument is inserted into the patient 140 to create the percutaneous access path. The control system 150 can provide information via the display(s) 152 regarding the catheter 130 to assist the physician 160 in navigating the catheter 130. For example, an interface(s) 154 can provide image data from the perspective of the scope 120. The image data may depict the catheter 130 (such as when within the FOV of an imaging device of the scope 120). In examples, the catheter 130 may be driven from a third-person perspective (such as from the viewpoint of the scope 120).

Once the scope 120 and/or the catheter 130 are located at the target location, the physician 160 can use the scope 120 to break up the kidney stone and/or use the catheter 130 to extract pieces of the kidney stone from the patient 140. For example, the scope 120 can deploy a tool (such as a laser, lithotripter, or cutting instrument) to fragment the kidney stone into pieces and the catheter 130 can suck out the pieces from the kidney through the percutaneous access path. In examples, the catheter 130 and/or the scope 120 can provide irrigation and/or aspiration to facilitate removal of the kidney stone. For instance, the catheter 130 can be coupled to an irrigation and/or aspiration system.

The medical system 100 can provide a variety of benefits, such as providing guidance to assist a physician in performing a procedure (such as instrument tracking, instrument navigation, instrument calibration, etc.), enabling a physician to perform a procedure from an ergonomic position without the need for awkward arm motions and/or positions, enabling a single physician to perform a procedure with one or more medical instruments, avoiding radiation exposure (such as associated with fluoroscopy techniques), enabling a procedure to be performed in a single-operative setting, providing continuous suction to remove an object more efficiently (such as to remove a kidney stone), and so on. For example, the medical system 100 can provide guidance information to assist a physician in using various medical instruments to access a target anatomical feature while minimizing bleeding and/or damage to anatomy (such as critical organs, blood vessels, etc.). Further, the medical system 100 can provide non-radiation-based navigational and/or localization techniques to reduce physician and patient exposure to radiation and/or reduce the amount of equipment in the operating room. Moreover, the medical system 100 can provide functionality that is distributed between at least the control system 150 and the robotic system 110, which can be independently movable. Such distribution of functionality and/or mobility can enable the control system 150 and/or the robotic system 110 to be placed at locations that are optimal for a particular medical procedure, which can maximize working area around the patient and/or provide an optimized location for a physician to perform a procedure.

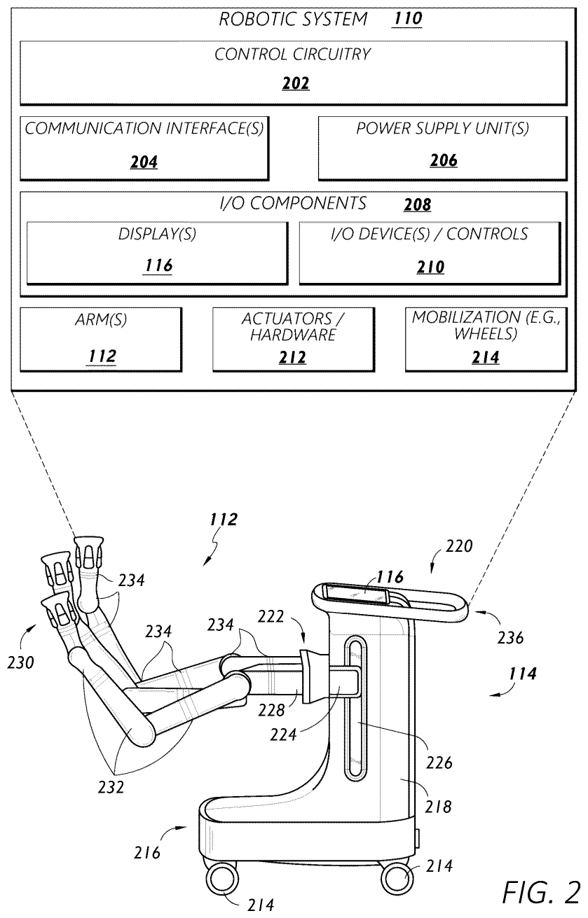

FIG. 2 shows an example of the robotic system 110 depicted in FIG. 1, according to some implementations. As shown, the robotic system 110 can include control circuitry 202, a communication interface(s) 204 (such as configured to communicate with one or more components/devices), a power supply unit(s) 206 (such as configured to supply/manage power to components of the robotic system 110), I/O components 208 (such as the display(s) 116 and/or other I/O devices/controls 210), the one or more robotic arms 112, actuators/hardware 212, and/or mobilization component(s) 214 (such as wheels). In some implementations, the robotic system 110 can comprise a housing/enclosure configured and/or dimensioned to house or contain at least part of one or more of the components of the robotic system 110. In this example, the robotic system 110 is illustrated as a cart-based system that is movable with the one or more wheels 214. In some cases, after reaching the appropriate position, the one or more wheels 214 can be immobilized using wheel locks to hold the robotic system 110 in place. However, the robotic system 110 can be implemented as a stationary system, integrated into another system/device, and so on.

In some implementations, one or more of the components of the robotic system 110 can be electrically and/or communicatively coupled using certain connectivity circuitry/devices/features, which can or may not be part of the control circuitry 202. For example, the connectivity feature(s) can include one or more printed circuit boards configured to facilitate mounting and/or interconnectivity of at least some of the various components/circuitry of the robotic system 110. Although certain components of the robotic system 110 are illustrated in FIG. 2, it should be understood that additional components not shown can be included in implementations in accordance with the present disclosure. Furthermore, certain of the illustrated components can be omitted in some implementations.

The support structure 114 can include a base 216, an elongated column 218, and/or a console 220 at the top of the column 218. The column 218 can include one or more arm supports 222 (also referred to as a “carriage”) for supporting the deployment of the one or more robotic arms 112 (three shown in FIG. 2). The arm support 222 can include individually-configurable arm mounts that rotate along a perpendicular axis to adjust the base of the robotic arms 112 for better positioning relative to a patient. The arm support 222 also includes a column interface 224 that allows the arm support 222 to vertically translate along the column 218. In some implementations, the column interface 222 can be connected to the column 218 through slots, such as slot 226, that are positioned on opposite sides of the column 218 to guide the vertical translation of the arm support 222. The slot 226 contains a vertical translation interface to position and hold the arm support 222 at various vertical heights relative to the base 216. Vertical translation of the arm support 222 allows the robotic system 110 to adjust the reach of the robotic arms 112 to meet a variety of table heights, patient sizes, and/or physician preferences. Similarly, the individually-configurable arm mounts on the arm support 222 can allow the base 216 to be angled in a variety of configurations.

The robotic arms 112 may generally comprise robotic arm bases 228 and end effectors 230, separated by a series of linkages 232 (also referred to as “arm segments”) that are connected by a series of joints 234, each joint comprising one or more independent actuators 212. Each actuator may comprise an independently-controllable motor. Each independently-controllable joint 234 can provide or represent an independent degree of freedom available to the robotic arm. In some implementations, each of the arms 112 has seven joints, and thus provides seven degrees of freedom, including “redundant” degrees of freedom. Redundant degrees of freedom allow the robotic arms 112 to position their respective end effectors 230 at a specific position, orientation, and/or trajectory in space using different linkage positions and joint angles. This allows for the robotic system 110 to position and direct a medical instrument from a desired point in space while allowing a physician to move the arm joints into a clinically advantageous position away from the patient to create greater access, while avoiding arm collisions.

Each end effector 230 can comprise an instrument device manipulator (IDM), which may be attached using a mechanism changer interface (MCI). In some implementations, the IDM can be removed and replaced with a different type of IDM, for example, a first type of IDM may manipulate an endoscope, while a second type of IDM may manipulate a laparoscope. The MCI can include connectors to transfer pneumatic pressure, electrical power, electrical signals, and/or optical signals from the robotic arm 112 to the IDM. The IDMs may be configured to manipulate medical instruments (such as surgical tools/instruments) using techniques including, for example, direct drive, harmonic drive, geared drives, belts and pulleys, magnetic drives, and the like.

The robotic system base 216 can balance the weight of the column 218, the arm support 222, and/or arms 112 over the floor. Accordingly, the robotic system base 216 can house heavier components, such as electronics, motors, power supply, as well as components that selectively enable movement or immobilize the robotic system 110. In examples, such as that illustrated in FIG. 2, the robotic system base 216 includes wheel-shaped casters 214 that allow for the robotic system to easily move around the room prior to a procedure. After reaching the appropriate position, the casters 214 can be immobilized using wheel locks to hold the robotic system 110 in place during the procedure.

Positioned at the upper end of column 218, the console 220 allows for both a user interface for receiving user input and the display screen 116 (or a dual-purpose device such as, for example, a touchscreen) to provide a physician/user with pre-operative and/or intra-operative data. Potential pre-operative data can include pre-operative plans, navigation and mapping data derived from pre-operative computerized tomography (CT) scans, and/or notes from pre-operative patient interviews. Intra-operative data can include optical information provided from the tool, sensor and coordinate information from sensors, as well as vital patient statistics, such as respiration, heart rate, and/or pulse. The console 220 can be positioned and tilted to allow a physician to access the console 220 from the side of the column 218 opposite the arm support 222. From this position, the physician may view the console 220, the robotic arms 112, and/or patient while operating the console 220 from behind the robotic system 110. As shown, the console 220 can also include a handle 236 to assist with maneuvering and stabilizing robotic system 110.

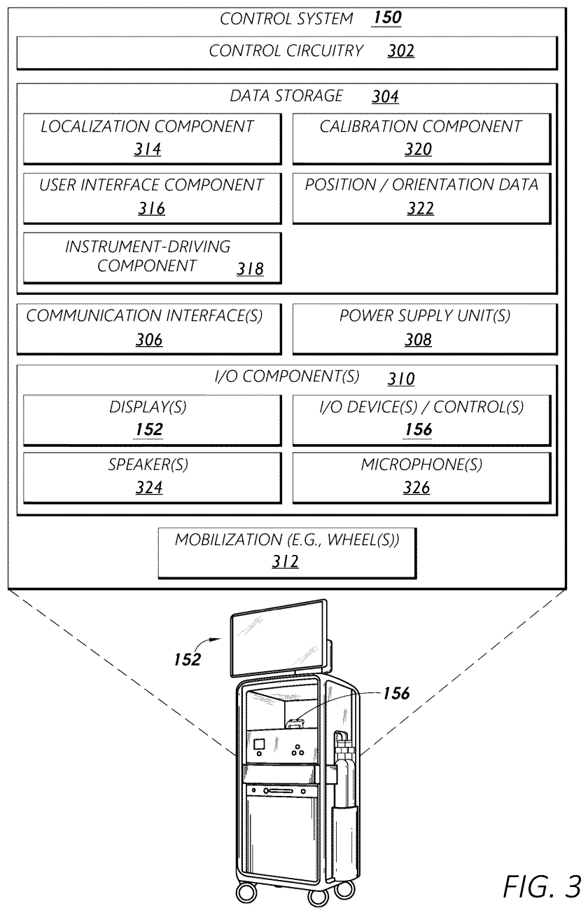

FIG. 3 illustrates example details of the control system 150 from FIG. 1 in accordance with one or more implementations. As illustrated, the control system 150 can include one or more of the following components, devices, modules, and/or units (referred to herein as “components”), either separately/individually and/or in combination/collectively: control circuitry 302, data storage/memory 304, one or more communication interfaces 306, one or more power supply units 308, one or more input/output (I/O) components 310, and/or mobilization 312 (such as casters or other types of wheels). In some implementations, the control system 150 can comprise a housing/enclosure configured and/or dimensioned to house or contain at least part of one or more of the components of the control system 150. In this example, the control system 150 is illustrated as a cart-based system that is movable with the one or more wheels 312. In some cases, after reaching the appropriate position, the one or more wheels 312 can be immobilized using wheel locks to hold the control system 150 in place. However, the control system 150 can be implemented as a stationary system, integrated into another system/device, and so on.

Although certain components of the control system 150 are illustrated in FIG. 3, it should be understood that additional components not shown can be included in implementations in accordance with the present disclosure. Furthermore, certain of the illustrated components can be omitted in some implementations. Although the control circuitry 302 is illustrated as a separate component in the diagram of FIG. 3, it should be understood that any or all of the remaining components of the control system 150 can be embodied at least in part in the control circuitry 302. That is, the control circuitry 302 can include various devices (active and/or passive), semiconductor materials and/or areas, layers, regions, and/or portions thereof, conductors, leads, vias, connections, and/or the like, wherein one or more of the other components of the control system 150 and/or portion(s) thereof can be formed and/or embodied at least in part in/by such circuitry components/devices.

The various components of the control system 150 can be electrically and/or communicatively coupled using certain connectivity circuitry/devices/features, which can or may not be part of the control circuitry 302. For example, the connectivity feature(s) can include one or more printed circuit boards configured to facilitate mounting and/or interconnectivity of at least some of the various components/circuitry of the control system 150. In some implementations, two or more of the control circuitry 302, the data storage/memory 304, the communication interface(s) 306, the power supply unit(s) 308, and/or the I/O component(s) 310, can be electrically and/or communicatively coupled to each other.

As illustrated, the memory 304 can include a localization component 314, a user interface component 316, an instrument-driving component 318, and a calibration component 320 configured to facilitate various functionality discussed herein. In some implementations, the localization component 314, the user interface component 316, the instrument-driving component 318, and/or the calibration component 320 can include one or more instructions that are executable by the control circuitry 302 to perform one or more operations. Although many implementations are discussed in the context of the components 314-320 including one or more instructions that are executable by the control circuitry 302, any of the components 314-320 can be implemented at least in part as one or more hardware logic components, such as one or more application specific integrated circuits (ASIC), one or more field-programmable gate arrays (FPGAs), one or more program-specific standard products (ASSPs), one or more complex programmable logic devices (CPLDs), and/or the like. Furthermore, although the components 314-320 are illustrated as being included within the control system 150, any of the components 314-320 can be implemented at least in part within another device/system, such as the robotic system 110, the table 170, or another device/system. Similarly, any of the other components of the control system 150 can be implemented at least in part within another device/system.

The localization component 314 can be configured to perform one or more localization techniques to determine and/or track a position and/or an orientation of an object, such as a medical instrument. For example, the localization component 314 can process input data (such as sensor data from a medical instrument, model data regarding anatomy of a patient, position data of a patient, pre-operative data, robotic command and/or kinematics data, etc.) to generate position/orientation data 322 for one or more medical instruments. In examples, the position/orientation data 322 can indicate a location and/or an orientation of one or more medical instruments relative to a frame of reference. The frame of reference can be a frame of reference relative to anatomy of a patient, a known object (such as an EM field generator, a robot arm, or another medical instrument), a coordinate system/space, a control frame, and so on. In some implementations, the position/orientation data 322 can indicate a location and/or an orientation of a distal end of a medical instrument (and/or proximal end, in some cases).

In some implementations, the localization component 314 can process pre-operative data to determine a position and/or an orientation of an object. The pre-operative data (sometimes referred to as “mapping data”) can be generated by performing CT scans, such as low dose CT scans. The pre-operative CT images from the scans can be reconstructed into three-dimensional images, which are visualized as “slices,” of a cutaway view of a patient's internal anatomy. When analyzed in the aggregate, image-based models for anatomical cavities, spaces, and/or structures of the patient's anatomy, such as a patient lung network, or the renal anatomy can be generated. A center-line geometry can be determined and/or approximated from the CT images to develop a three-dimensional volume of the patient's anatomy, referred to as model data (also referred to as “pre-operative model data” when generated using only pre-operative CT scans). Network topological models can also be derived from CT-images.

Further, in some implementations, the localization component 314 can perform vision-based techniques to determine a position and/or an orientation of an object. For example, a medical instrument can be equipped with a camera, a range sensor (sometimes referred to as “a depth sensor”), or a radar device, to provide sensor data in the form of vision data. The localization component 314 can process the vision data to facilitate vision-based location tracking of the medical instrument. For example, a pre-operative model data can be used in conjunction with vision data to enable computer vision-based tracking of a medical instrument (such as an endoscope). In examples, using pre-operative model data, the control system 150 can generate a library of expected endoscopic images based on the expected path of travel of a scope, with each image being linked to a location within the model. Intra-operatively, this library can be referenced by the control system 150 in order to compare real-time images and/or other vision data captured at a scope (such as a camera at a distal end of an endoscope) to those in the image library to assist with localization.

Moreover, in some implementations, other types of vision-based techniques can be performed to determine a position and/or an orientation of an object. For example, the localization component 314 can use feature tracking to determine motion of an image sensor (such as a camera or other sensor), and thus, a medical instrument associated with the image sensor. In some cases, the localization component 314 can identify circular geometries in pre-operative model data that correspond to anatomical lumens and track the change of those geometries to determine which anatomical lumen was selected, as well as the relative rotational and/or translational motion of the medical instrument. Use of a topological map can also enhance vision-based algorithms or techniques. Furthermore, the localization component 314 can use optical flow, another computer vision-based technique, to analyze displacement and/or translation of image pixels in a video sequence in vision data to infer camera movement. Examples of optical flow techniques can include motion detection, object segmentation calculations, luminance, motion compensated encoding, stereo disparity measurement, etc. By comparing multiple frames over multiple iterations, the localization component 314 can determine movement and a location of an image sensor (and thus an endoscope).

Furthermore, in some implementations, the localization component 314 can use electromagnetic tracking to determine a position and/or an orientation of an object. For example, the localization component 314 can use real-time EM tracking to determine a real-time location of a medical instrument in a coordinate system/space (and/or relative to another medical instrument) that can be registered to the patient's anatomy, which can be represented by a pre-operative model or other model. In EM tracking, an EM sensor (or tracker) including one or more sensor coils can be embedded in one or more locations and/or orientations in a medical instrument (such as a scope, a needle, etc.). The EM sensor can measure a variation in an EM field created by one or more static EM field generators positioned at a known location. The location information detected by the EM sensors can be stored as EM data. The localization component 314 can process the EM data to determine a position and/or orientation of an object, such as a medical instrument.

An EM field generator (or transmitter) can be placed close to the patient (such as within a predetermined distance) to create a low intensity magnetic field that an EM sensor can detect. The magnetic field can induce small currents in the sensor coils of the EM sensor, which can be analyzed to determine a distance and/or angle between the EM sensor and the EM field generator. These distances and/or orientations can be intra-operatively “registered” to patient anatomy (such as a pre-operative model) in order to determine a geometric transformation that aligns a single location in a coordinate system with a position in the pre-operative model of the patient's anatomy. Once registered, an EM sensor (such as an embedded EM tracker) in one or more positions of a medical instrument (such as the distal tip of an endoscope, a needle, etc.) can provide real-time indications of a position and/or an orientation the medical instrument through the patient's anatomy.

Additionally, or alternatively, in some implementations, the localization component 314 can use robotic command and/or kinematics data to determine a position and/or an orientation of an object. Robotic command and/or kinematics data can be indicative of position/orientation of a robotic arm (such as pitch and/or yaw) resulting from an articulation command, such as those used during pre-operative calibration and/or during a procedure. In examples, the localization component 314 can use data indicative of a position/orientation of a robotic arm to determine a position/orientation of a medical instrument attached to the robotic arm. For instance, based on a position/orientation of a robotic arm that is attached to a catheter, command sent to control the catheter, and/or a characteristic(s) of the catheter (such as a length of the catheter and/or capabilities of the catheter), the localization component 314 can determine/estimate a position/orientation of the catheter.

Further, in examples, the localization component 314 can use robotic command data to determine how far a medical instrument has been inserted/retracted, such as within a patient, based on commands to control the medical instrument, markings on the medical instrument indicating distance, etc. In some intra-operatively implementations, calibration measurements can be used in combination with known insertion depth information to estimate a position and/or an orientation of a medical instrument. Alternatively, or additionally, these calculations can be analyzed in combination with EM, vision, and/or topological modeling to estimate a position and/or orientation of a medical instrument.

Further, in some implementations, the localization component 314 can use other types of data to determine a position and/or an orientation of an object. For example, the localization component 314 can analyze sensor data from a shape sensing fiber (such as which can provide shape data regarding a location/shape of a medical instrument), an accelerometer, a gyroscope, a satellite-based positioning sensor (such as a global positioning system (GPS)), a radio-frequency transceiver, and so on, embedded on a medical instrument. Such data can be indicative of a position and/or an orientation of the medical instrument.

In some implementations, the localization component 314 can use input data in combination. For example, the localization component 314 can use a probabilistic approach where a confidence weight is assigned to a position/orientation determined from multiple forms of input data. To illustrate, if EM data is not as reliable (as may be the case where there is EM interference), the EM data can be associated with a relatively low confidence value and other forms of input data can be relied on, such as vision data, robotic command and kinematics data, and so on.

The user interface component 316 can be configured to facilitate one or more user interfaces (also referred to as “graphical user interfaces” or “GUIs”). For example, the user interface component 316 can generate user interface data representing one or more of the interfaces described herein. The user interface component 316 can present one or more visualizations or other information to assist in driving a medical instrument and/or controlling the robotic system 110. In examples, the user interface component 316 can generate a visual representation of image data captured by a scope. The user interface component 316 can provide user interface data or other data to the one or more displays 156 and/or another display(s) for display of information.

The instrument-driving component 318 can be configured to drive a medical instrument. For example, the instrument-driving component 318 can be configured to process directional input signals from the I/O device(s) 156, process position or/orientation data 322 regarding a medical instrument, generate control signals, send the control signals to the robotic system 110 to control movement of an instrument(s) connected to the robotic system 110, and so on. In some implementations, the instrument-driving component 318 can facilitate driving of a medical instrument from a perspective of another medical instrument. Further, in some implementations, the instrument-driving component 318 can facilitate one or more driving/control modes to assist a physician in driving a medical instrument, such as a direct control mode, an inverted control mode, and so on.

The calibration component 320 can be configured to calibrate a control scheme/control frame of reference for a medical instrument. For example, the calibration component 320 can determine an orientation of a first instrument relative to a second instrument, such as an orientation of a distal end of a catheter relative to a distal end of a scope. Based on the orientation of the instruments, the calibration component 320 can adjust a control scheme associated with controlling the first/second instrument.

Although not illustrated in FIG. 3, in some implementations, the data storage 304 can include a target component configured to determine a position of a target location within the human anatomy and/or a coordinate space/system. A target location can represent a point/point set within the human anatomy and/or a coordinate space/system. For example, the target component can identify one or more points for a target location within a coordinate system, identify coordinates for the one or more points (such as X, Y, Z coordinates for each point), and associate the coordinates with the target location. In some implementations, the target component can use a position and/or orientation of a medical instrument to determine a position of a target location. For example, a scope can be navigated to contact or be within proximity to a target location (such as parked in-front of the target location). The localization component 314 can use localization techniques to determine a position of the scope (such as a location of the end of the scope) and/or a position of an object within a field-of-view (FOV) of the scope. The target component can associate the position of the scope (such as the coordinates of the scope) with the target location. Additionally, or alternatively, in some implementations, a scope can deliver a fiduciary to mark a target location and a position of the fiduciary can be determined.

A target location can represent a fixed or movable point(s) within the human anatomy and/or a coordinate space/system. For example, if a papilla is initially designated as a target location, coordinates for the target location can be determined and updated as the procedure proceeds and the papilla moves (such as due to insertion of a medical instrument). Here, a location of a scope (which can be within proximity to the papilla) can be tracked over time and used to update the coordinates of the target location. In some implementations, the target component can estimate/predict a position of a target location. Here, the target location can be represented with the predicted position. For example, the target component can use an algorithm to predict coordinates of the target location as the human anatomy moves. The predicted coordinates can be used to determine a target trajectory.

The one or more communication interfaces 306 can be configured to communicate with one or more device/sensors/systems. For example, the one or more communication interfaces 306 can send/receive data in a wireless and/or wired manner over a network. A network in accordance with implementations of the present disclosure can include a local area network (LAN), wide area network (WAN) (such as the Internet), personal area network (PAN), body area network (BAN), etc. In some implementations, the one or more communication interfaces 306 can implement a wireless technology such as Bluetooth, Wi-Fi, near field communication (NFC), or the like.

The one or more power supply units 308 can be configured to manage power for the control system 150 (and/or the robotic system 110, in some cases). In some implementations, the one or more power supply units 308 (and/or any other power supply unit) can include one or more batteries, such as a lithium-based battery, a lead-acid battery, an alkaline battery, and/or another type of battery. That is, the one or more power supply units 308 can comprise one or more devices and/or circuitry configured to provide a source of power and/or provide power management functionality. Moreover, in some implementations, the one or more power supply units 308 include a mains power connector that is configured to couple to an alternating current (AC) or direct current (DC) mains power source.

The one or more I/O components 310 can include a variety of components to receive input and/or provide output, such as to interface with a user. The one or more I/O components 310 can be configured to receive touch, speech, gesture, or any other type of input. In examples, the one or more I/O components 310 can be used to provide input regarding control of a device/system, such as to control the robotic system 110, navigate a medical instrument attached to the robotic system 110, control the table 170, control a fluoroscopy device, and so on. As shown, the one or more I/O components 310 can include the one or more displays 152 (sometimes referred to as “the one or more display devices 152”) configured to display data. The one or more displays 152 can include one or more liquid-crystal displays (LCD), light-emitting diode (LED) displays, organic LED displays, plasma displays, electronic paper displays, and/or any other type(s) of technology. In some implementations, the one or more displays 152 include one or more touchscreens configured to receive input and/or display data. Further, the one or more I/O components 310 can include the one or more I/O devices/controls 156, which can include a touchscreen, touch pad, controller, mouse, keyboard, wearable device (such as optical head-mounted display), virtual or augmented reality device (such as head-mounted display), etc. Additionally, the one or more I/O components 310 can include one or more speakers 324 configured to output sounds based on audio signals and/or one or more microphones 326 configured to receive sounds and generate audio signals. In some implementations, the one or more I/O components 310 include or are implemented as a console.

Although not shown in FIG. 3, the control system 150 can include and/or control other components, such as one or more pumps, flow meters, valve controls, and/or fluid access components in order to provide controlled irrigation and/or aspiration capabilities to a medical instrument (such as a scope), a device that can be deployed through a medical instrument, and so on. In some implementations, irrigation and/or aspiration capabilities can be delivered directly to a medical instrument through separate cable(s). Further, the control system 150 can include a voltage and/or surge protector designed to provide filtered and/or protected electrical power to another device, such as the robotic system 110, thereby avoiding placement of a power transformer and other auxiliary power components in robotic system 110, resulting in a smaller, more moveable robotic system 110.

The control system 150 can also include support equipment for sensors deployed throughout the medical system 100. For example, the control system 150 can include opto-electronics equipment for detecting, receiving, and/or processing data received from optical sensors and/or cameras. Such opto-electronics equipment can be used to generate real-time images for display in any number of devices/systems, including in the control system 150. Similarly, the control system 150 can include an electronic subsystem for receiving and/or processing signals received from deployed electromagnetic (EM) sensors. In some implementations, the control system 150 can also be used to house and position an EM field generator for detection by EM sensors in or on a medical instrument.

In some implementations, the control system 150 can be coupled to the robotic system 110, the table 170, a medical instrument, etc., through one or more cables or connections (not shown). In some implementations, support functionality from the control system 150 can be provided through a single cable, simplifying and de-cluttering an operating room. In other implementations, specific functionality can be coupled in separate cabling and connections. For example, while power can be provided through a single power cable, the support for controls, optics, fluidics, and/or navigation can be provided through a separate cable.

The term “control circuitry” (such as the control circuitry 202, the control circuitry 302, and/or any other control circuitry) is used herein according to its broad and ordinary meaning, and can refer to any collection of one or more processors, processing circuitry, processing modules/units, chips, dies (such as semiconductor dies including come or more active and/or passive devices and/or connectivity circuitry), microprocessors, micro-controllers, digital signal processors, microcomputers, central processing units, graphics processing units, field programmable gate arrays, programmable logic devices, state machines (such as hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in implementations in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

One or more computer-readable media that can be implemented in accordance with implementations of the present disclosure includes, but is not limited to, phase change memory, static random-access memory (SRAM), dynamic random-access memory (DRA M), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

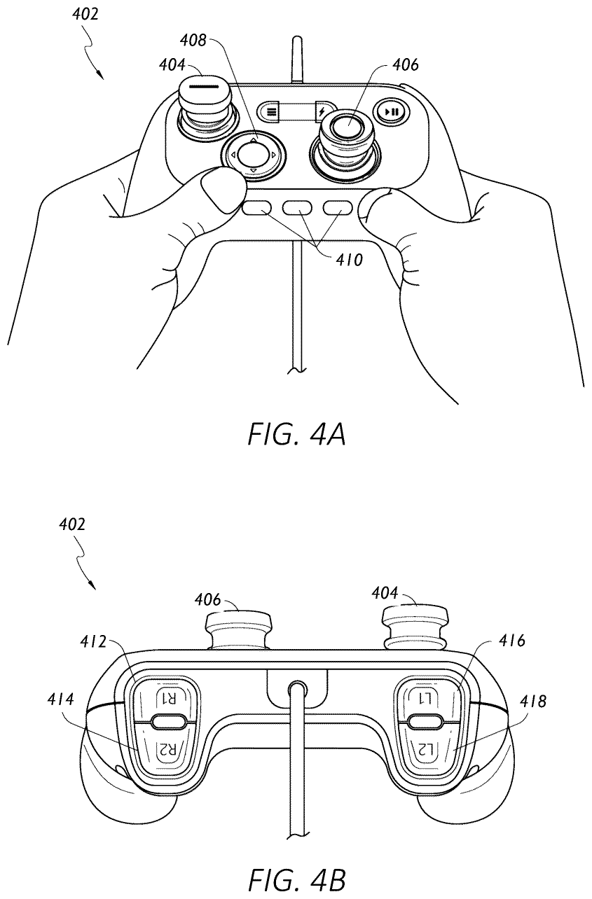

FIGS. 4A and 4B illustrate example details of a controller 402 in accordance with one or more implementations. In examples, the I/O device(s) 156 of the control system 150 and/or another I/O device discussed herein is implemented as the controller 402. However, the I/O device(s) 156 can be implemented as other types of devices. FIG. 4A and FIG. 4B illustrates a perspective view and a side view of the controller 402, respectively, according to certain implementations.

The controller 402 can receive/facilitate axis movement inputs, such via one or more joysticks 404, 406 and/or one or more directional pads 408. For example, a user can manipulate the one or more joysticks 404, 406 (and/or the one or more directional pads 408, in some cases) to provide directional input to control a medical instrument. In some implementations, the joysticks 404, 406 provide analog input while the directional pad 408 provides digital input. However, any of the joysticks 404, 406 and/or the directional pad 408 can provide analog and/or digital input. In examples, input received via the one or more directional pads 408 can be used to control a user interface, while input received via the one or more joysticks 404, 406 can be used to control movement of a medical instrument. The controller 402 can further include buttons 410 to provide additional control input. In the example illustrated in FIG. 4B, the controller 402 includes four buttons on the side of the controller: “RI” 412, “R2” 414, “LI” 416, and “L2” 418. Other implementations can include a different number of buttons and/or a different layout. In some implementations, the controller 402 can be a game-type console controller (and/or similar to a game-type console controller) repurposed to work with the control system 150. For example, controller game firmware may be overwritten with a medical device firmware and/or an input device manager can be installed in a component of the medical system 100 (such as the control system 150) to convert inputs from the controller 402 into inputs understandable by the robotic system 110.

The controller 402 can be implemented to receive input to control/drive a medical instrument. For example, the joysticks 404, 406 can receive directional input indicative of a direction to move a medical instrument (such as right, left, diagonal, up, down, insert, or retract). To illustrate, a user can tilt the joystick 406 to the left/right to cause a catheter/scope to move in a left/right direction (which can depend on a control mode) relative to a control frame, as discussed above. In another illustrate, a user can push/tilt the joystick 404 forward/back relative to FIG. 4A to cause a catheter/scope to be inserted/retracted (depending on a control mode). Although certain controls are discussed as mapping to certain functionality, the controller 402 can be configured in a variety of other manners. In some implementations, the controller 402 can be customized with a user interface that allows assigning of functionality to a particular control on the controller 402.

In some implementations, the controller 402 can implement a control (such as one or more of the controls 404-418 and/or other controls) to facilitate switching between different medical instruments. For example, a user can select one of the buttons 410 to switch from driving a scope to driving a catheter. Further, the controller 402 can implement a control to switch between control/driving modes for a medical instrument(s), such as a direct control mode, an inverted control mode, etc. Moreover, the controller 402 can implement a control to navigate to a specific interface, such as a driving interface, a calibration interface.

As described with reference to FIG. 1, the catheter 130 is often driven from a third-person perspective (such as from the FOV of a camera disposed on or proximate to the distal end of the scope 120). As a result, some users may struggle to understand how directional inputs (such as tilting the joystick 406 up, down, left, or right) translate to movements by the catheter 130. For example, many existing catheters are cylindrical in shape and rotationally symmetric about the roll axis (such that the pitch and yaw axes orthogonal to the roll axis are virtually indistinguishable from a third-person perspective). Thus, a user may need to test various input directions and see how a catheter responds. This trial-and-error approach to navigation can be frustrating to the user while prolonging the duration of the procedure. Aspects of the present disclosure recognize that such guesswork can be eliminated by marking or labeling one or more sides of a catheter so that the catheter appears asymmetric about its roll axis. Example suitable markings may include any visual cue or indication that reduces or eliminates the rotational symmetry of the catheter (such as colors, shapes, lights, patterns, and/or various other features that can be seen in the FOV of the scope camera or displayed on the interface 154).

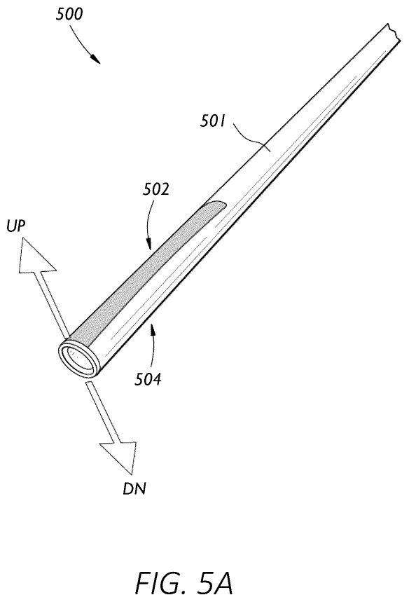

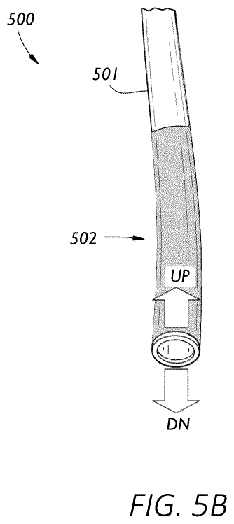

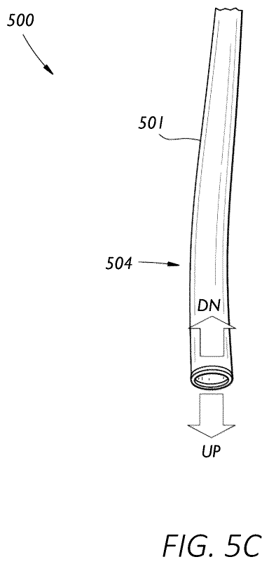

FIGS. 5A-5C show an example medical instrument 500, according to some implementations. More specifically, FIG. 5A shows a perspective view of the medical instrument 500, FIG. 5B shows a top view of the medical instrument 500, and FIG. 5C shows a bottom view 520 of the medical instrument 500. The medical instrument 500 can be any instrument having an elongate shaft 501 that is symmetrical in shape about its roll axis. In some implementations, the medical instrument 500 may be one example of the catheter 130 of FIG. 1.

The elongate shaft 501 has a top side 502 and a bottom side 504 associated with up and down movements, respectively, (or pitch) of the shaft 501. For example, directional inputs in the up direction (such as by tilting the joystick 406 up) may cause the distal end of the shaft 501 to pitch or deflect in a direction pointing radially towards the top side 502 of the shaft 501 (as indicated by the “UP” arrow). By contrast, directional inputs in the down direction (such as by tilting the joystick 406 down) may cause the distal end of the shaft 501 to pitch or deflect in a direction pointing radially towards the bottom side 504 of the shaft 501 (as indicated by the “DN” arrow). In some implementations, the top side 502 may be painted a different color and/or shade than the bottom side 504, which provides a visual feature on the outer surface of the shaft 501 that differentiates the top side 502 from the bottom side 504 of the shaft 501. In the example of FIGS. 5A-5C, the top side 502 is shown painted a darker color (such as gray) while the remainder of the shaft 501 is shown painted a lighter color (such as white).

The color schemes associated with the different sides of the shaft 501 can be used to visualize how the shaft 501 will move in response to various directional inputs, regardless of how the shaft 501 is oriented with respect to the FOV of a scope (or other third-person perspective). With reference for example to FIG. 5A, a user may discern, based on the exposed colors of the top side 502 and the bottom side 504, that directional inputs in the up direction will cause the distal end of the shaft 501 to articulate towards the upper-left corner of the page. With reference for example to FIG. 5B, a user may discern, based on the exposed color of the top side 502 (where the bottom side 504 is hidden from view), that directional inputs in the up direction will cause the distal end of the shaft 501 to articulate out of the page. With reference for example to FIG. 5C, a user may discern, based on the exposed color of the bottom side 504 (where the top side 502 is hidden from view), that directional inputs in the up direction will cause the distal end of the shaft 501 to articulate into the page.

In the example of FIGS. 5A-5C, two color tones are used to distinguish the top side 502 of the shaft 501 from the bottom side 504 of the shaft 501 (to indicate a direction of pitch). In some other implementations, the two color tones may be mapped to the left and/or right sides of the shaft 501 (to indicate a direction of yaw). Still further, in some implementations, three or more color tones may be used to distinguish the top side 502 from the bottom side 504 as well as to distinguish the left side from the right side of the shaft 501. In some aspects, to further reduce the cognitive load on the user, a combination of colors and patterns (or designs) may be used to distinguish the four cardinal directions (such as up, down, left, and right) associated with an instrument. For example, different colors can be mapped to the top side 502 and the bottom side 504 whereas different shapes and/or patterns can be mapped to the left and right sides of the shaft 501. In some implementations, a graphical interface may be presented to the user (such as the interface 154 of FIG. 1) to indicate the mapping between the various visual indicators (such as colors and/or patterns) and the associated directions of movement of the shaft 501.

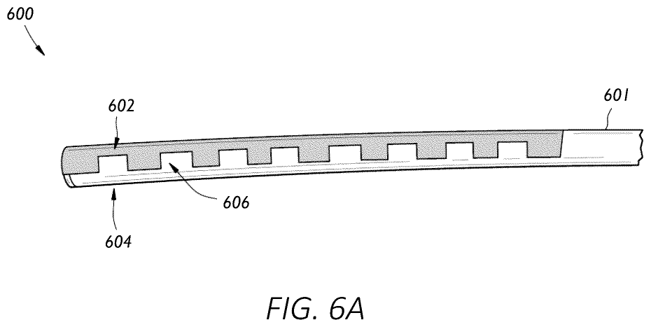

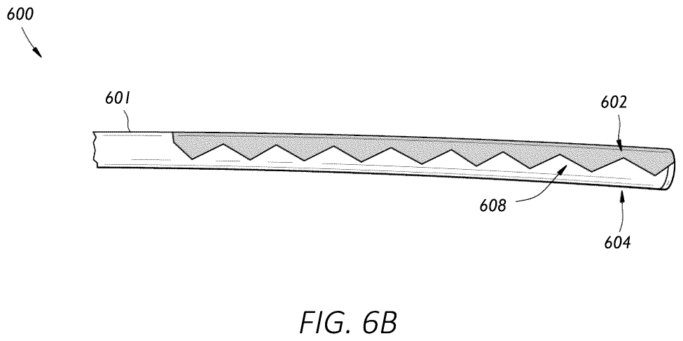

FIGS. 6A and 6B shows another example medical instrument 600, according to some implementations. More specifically, FIG. 6A shows a side view of the medical instrument 600 (where the distal end of the instrument is pointing to the left) and FIG. 6B shows another side view of the medical instrument 600 (where the distal end of the instrument is pointing to the right). The medical instrument 600 can be any instrument having an elongate shaft 601 that is symmetrical in shape about its roll axis. In some implementations, the medical instrument 600 may be one example of the catheter 130 of FIG. 1.

The elongate shaft 601 has a top side 602, a bottom side 604, a left side 606, and a right side 608 associated with up, down, left, and right movements, respectively, of the shaft 601. For example, directional inputs in the up direction (such as by tilting the joystick 406 up) may cause the distal end of the shaft 601 to pitch or deflect in a direction pointing radially towards the top side 602 of the shaft 601, whereas directional inputs in the down direction (such as by tilting the joystick 406 down) may cause the distal end of the shaft 601 to pitch or deflect in a direction pointing radially towards the bottom side 604 of the shaft 601. Similarly, directional inputs in the left direction (such as by tilting the joystick 406 to the left) may cause the distal end of the shaft 601 to yaw or deflect in a direction pointing radially towards the left side 606 of the shaft 601, whereas direction inputs in the right direction (such as by tilting the joystick 406 to the right) may cause the distal end of the shaft 601 to yaw or deflect in a direction pointing radially towards the right side 608 of the shaft 601.

In some implementations, the top side 602 may be painted a different color and/or shade than the bottom side 604, which provides a visual feature on the outer surface of the shaft 601 that differentiates the top side 602 from the bottom side 604 of the shaft 601. In some implementations, the junction or intersection between the different colors may have a different design or pattern on one side compared to other side, which provides another visual feature on the outer surface of the shaft 601 that further differentiates the left side 606 from the right side 608 of the shaft 601. In the example of FIGS. 6A and 6B, the top side 602 is shown painted a darker color (such as gray) while the remainder of the shaft 601 is shown painted a lighter color (such as white). As shown in FIG. 6A, the colors intersect in a square saw-tooth pattern along the left side 606 of the shaft 601. As shown in FIG. 6B, the colors intersect in a triangular saw-tooth pattern on the right side 608 of the shaft 601. Thus, the colors can be used to visualize how the shaft 601 will pitch and the patterns can be used to visualize how the shaft 601 will yaw in response to various directional inputs (such as described with reference to FIGS. 5A-5C).

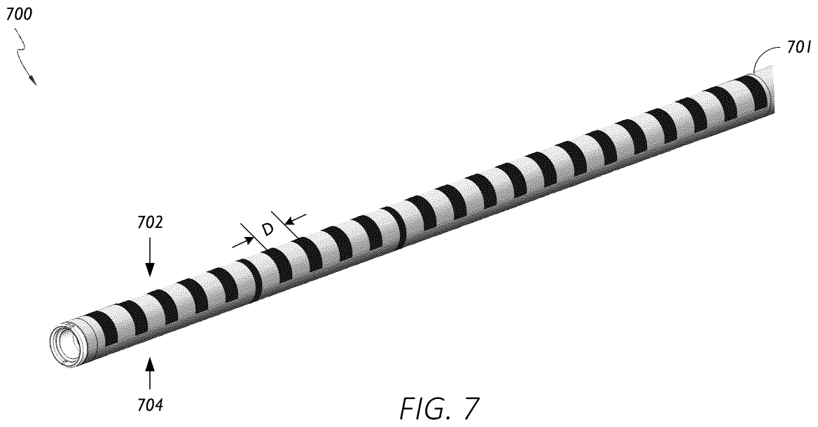

FIG. 7 shows another example medical instrument 700, according to some implementations. More specifically, FIG. 7 shows a perspective view of the medical instrument 700. The medical instrument 700 can be any instrument having an elongate shaft 701 that is symmetrical in shape about its roll axis. In some implementations, the medical instrument 700 may be one example of the catheter 130 of FIG. 1.

The elongate shaft 701 has a top side 702 and a bottom side 704 associated with up and down movements, respectively, of the shaft 701. For example, directional inputs in the up direction (such as by tilting the joystick 406 up) may cause the distal end of the shaft 701 to pitch or deflect in a direction pointing radially towards the top side 702 of the shaft 701, whereas directional inputs in the down direction (such as by tilting the joystick 406 down) may cause the distal end of the shaft 701 to pitch or deflect in a direction pointing radially towards the bottom side 704 of the shaft 701. Similarly, directional inputs in the left direction (such as by tilting the joystick 406 to the left) may cause the distal end of the shaft 701 to yaw or deflect in a direction pointing radially towards the left side of the shaft 701, whereas direction inputs in the right direction (such as by tilting the joystick 406 to the right) may cause the distal end of the shaft 701 to yaw or deflect in a direction pointing radially towards the right side of the shaft 701 (such as described with reference to FIGS. 6A and 6B).

In some aspects, the top side 702 may be painted with a series of stripes and/or patterns which provide a visual feature on the outer surface of the shaft 701 that differentiates the top side 702 from the bottom side 704 of the shaft 701. In some implementations, the stripes may be separated by a fixed or predetermined distance (D), which provides a frame of reference or scale for measuring size and/or distance in the FOV of a scope (such as the scope 120 of FIG. 1). For example, a user can estimate an insertion depth of the instrument 700 by counting the stripes painted on the top side 702 of the shaft 701. A user can also estimate the sizes of objects proximate to the shaft 701, for example, by comparing such objects to the stripes painted on the top side 702. In the example of FIG. 7, the stripes are shown painted in a darker color (such as black) while the remainder of the shaft 701 is shown painted a lighter color (such as gray). In some implementations, the ends of each stripe (such as at the intersection or junction between the top side 702 and the bottom side 704) can have a different pattern on the left side compared to the right side, for example, to provide a visual feature that differentiates the left side of the shaft 701 from the right side of the shaft 701 (such as described with reference to FIGS. 6A and 6B).

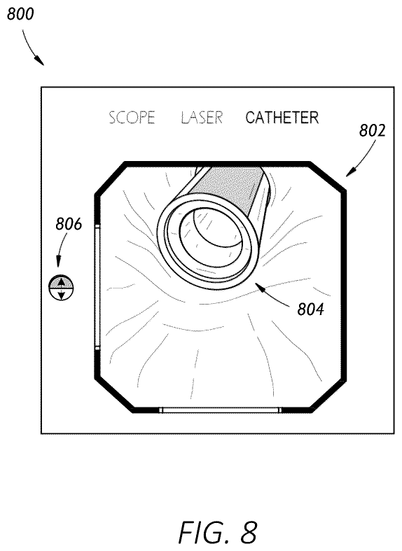

FIG. 8 shows an example graphical interface 800 for controlling a medical instrument, according to some implementations. In some implementations, the graphical interface 800 may be one example of the interface 154 of FIG. 1. More specifically, the graphical interface 800 may be displayed to a user of a medical system (such as the medical system 100 of FIG. 1) while the user is navigating a catheter 804 within an anatomy.

The graphical interface 800 is shown to include an image 802 depicting an FOV of a camera disposed on or proximate to the distal end of a scope (such as the scope 120). The image 802 shows a third-person perspective of the catheter 804 in relation to the scope and the surrounding anatomy. In some implementations, one or more sides of the catheter 804 may be marked or colored differently than the other sides of the catheter 804. In the example of FIG. 8, a top side of the catheter 804 is marked with a darker color (such as gray) and a bottom side of the catheter 804 is marked with a lighter color (such as white). The graphical interface 800 further includes a visual indicator 806 indicating a mapping of the colors to different directional inputs. As shown in FIG. 8, the visual indicator 806 shows the darker color being associated with an up arrow and the lighter color being associated with a down arrow. This indicates to the user that directional inputs in the up direction will cause the catheter 804 to move or deflect towards the darker marking and that direction inputs in the down direction will cause the catheter 804 to move or deflect towards the lighter marking (such as described with reference to FIGS. 5A-6). In some implementations, the visual indicator 806 also may indicate colors and/or patterns associated with left and right directional inputs (not shown for simplicity).

Aspects of the present disclosure described with reference to FIGS. 5A-8 can be implemented with minimal modification to existing medical instruments and/or systems. In some other aspects, image processing or computer vision techniques can be used to determine how a catheter will move in response to various directional inputs. In some other aspects, a camera may be disposed on the distal end of the catheter to enable a user to drive the catheter in a first-person perspective (similar to the scope). Still further, in some aspects, one or more sensors (such as EM sensors) may be disposed on the tip of a catheter and used to track the position and orientation of the catheter, in a world coordinate frame, relative to the position and orientation of a scope. The sensor information from both the scope and the catheter can be used to create a visual overlay for the command frame of the catheter. For example, the directionality of movement of the catheter can be mapped directly onto the image of the catheter in the FOV of the scope.

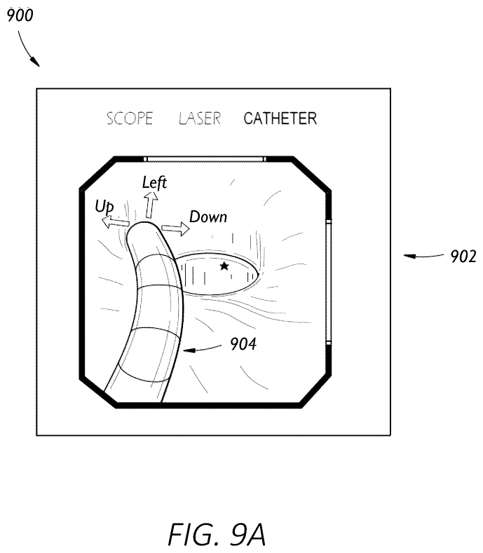

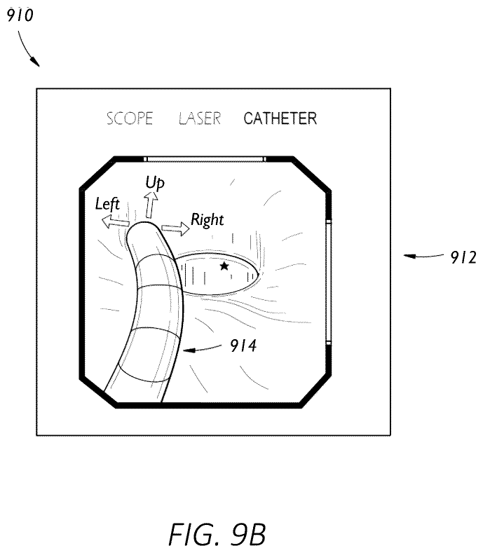

FIG. 9A shows another example graphical interface 900 for controlling a medical instrument, according to some implementations. In some implementations, the graphical interface 900 may be one example of the interface 154 of FIG. 1. More specifically, the graphical interface 900 may be displayed to a user of a medical system (such as the medical system 100 of FIG. 1) while the user is navigating a catheter 904 within an anatomy.

The graphical interface 900 is shown to include an image 902 depicting an FOV of a camera disposed on or proximate to the distal end of a scope (such as the scope 120). The image 902 shows a third-person perspective of the catheter 904 in relation to the scope and the surrounding anatomy. In some implementations, the medical system (such as the control circuitry 202 of FIG. 2 and/or the control circuitry 302 of FIG. 2) may determine a position and orientation of the catheter 904 in relation to a position and orientation of the camera and/or scope based on sensors disposed on or proximate to each of the instruments. The medical system uses the relative position and orientation of the catheter 904 to determine how the catheter 904 will respond to directional inputs based on the third-person perspective depicted by the image 902.

In the example of FIG. 9A, the graphical interface 900 is shown to include a visual indication (in the form of arrows and direction inputs, such as “up,” “down,” and “left”) overlaid upon the catheter 904. The arrows indicate the direction in which the catheter 904 will move in response to each of the inputs. For example, a directional input in the up direction may cause the catheter 904 to move towards the left of the image 902, a direction input in the down direction may cause the catheter 904 to move towards the right of the image 902, and a direction input in the left direction may cause the catheter 904 to move towards the top of the image 902.

In some other aspects, the medical system may use the relative position and orientation of the catheter 904 to adjust the command frame of the suction catheter 904 so that the controls feel instinctive regardless of the relative orientation of the instruments. For example, the medical system may provide a control scheme where a particular type of input (such as pushing on the joystick 406 of FIG. 4) always moves the tip of the catheter 904 towards (or away) from the tip of the scope.

FIG. 9B shows another example graphical interface 910 for controlling a medical instrument, according to some implementations. In some implementations, the graphical interface 910 may be one example of the interface 154 of FIG. 1. More specifically, the graphical interface 910 may be displayed to a user of a medical system (such as the medical system 100 of FIG. 1) while the user is navigating a catheter 914 within an anatomy.

The graphical interface 910 is shown to include an image 912 depicting an FOV of a camera disposed on or proximate to the distal end of a scope (such as the scope 120). The image 912 shows a third-person perspective of the catheter 914 in relation to the scope and the surrounding anatomy. In some implementations, the medical system (such as the control circuitry 202 of FIG. 2 and/or the control circuitry 302 of FIG. 3) may determine a position and orientation of the catheter 914 in relation to a position and orientation of the camera and/or scope based on sensors disposed on or proximate to each of the instruments. The medical system uses the relative position and orientation of the catheter 914 to determine the response of the catheter 914 to directional inputs based on the third-person perspective depicted by the image 912.

In the example of FIG. 9B, the graphical interface 910 is shown to include a visual indication (in the form of arrows and direction inputs, such as “up,” “down,” and “left”) overlaid upon the catheter 914. The arrows indicate the direction in which the catheter 914 will move in response to each of the inputs. In contrast to the mapping shown in FIG. 9A, the directional inputs shown in FIG. 9B are locked to a coordinate frame of the camera in the graphical interface 910. In other words, the medical system may adjust or remap the directional inputs to robotic commands for manipulating the catheter 914 so that directional inputs in the up direction always cause the catheter 914 to move towards the top of the image 912, directional inputs in the left direction always cause the catheter 914 to move towards the left of the image 912, and directional inputs in the right direction always causes the catheter 914 to move towards the right of the image 912, regardless of how the catheter 914 is oriented in the camera's FOV.

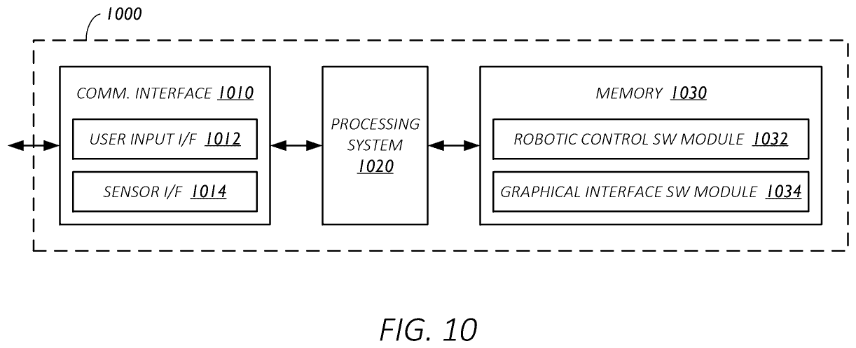

FIG. 10 shows a block diagram of an example system 1000 for controlling a medical instrument, according to some implementations. In some implementations, the system 1000 may be one example of the control circuitry 202 of FIG. 2 and/or the control circuitry 302 of FIG. 3. More specifically, the system 1000 is configured to control a robotic arm coupled to a medical instrument having an elongate shaft (such as the catheter 130 of FIG. 1).

The system 1000 includes a communication interface 1010, a processing system 1020, and a memory 1030. The communication interface 1010 is configured to communicate with one or more components of a medical system. More specifically, the communication interface 1010 includes a user input interface (I/F) 1012 for communicating with one or more user input devices (such as the controller 402 of FIGS. 4A and 4B) and a sensor interface (I/F) 1014 for communicating with one or more sensors (such as EM sensors or other sensors disposed on or proximate to various medical instruments). In some implementations, the user input I/F 1012 may receive user input for articulating a distal end of the elongate shaft in a plurality of directions orthogonal to its roll axis. In some implementations, the sensor I/F 1014 may receive first sensor data indicating a pose of the medical instrument and second sensor data indicating a pose of a camera disposed on another medical instrument (such as a scope).

The memory 1030 may include a non-transitory computer-readable medium (including one or more nonvolatile memory elements, such as EPROM, EEPROM, Flash memory, or a hard drive, among other examples) that may store the following software (SW) modules: a robotic control SW module 1032 to control the robotic arm to manipulate the medical instrument based on the received user inputs; and a graphical interface SW module 1034 to display a graphical interface including an image depicting the medical instrument in a field-of-view (FOV) of the camera and a visual indicator indicating a mapping of the plurality of directions to the medical instrument in the FOV of the camera. Each of the software modules 1032 and 1034 includes instructions that, when executed by the processing system 1020, causes the system 1000 to perform the corresponding functions.

The processing system 1020 may include any suitable one or more processors capable of executing scripts or instructions of one or more software programs stored in the system 1000 (such as in the memory 1030). For example, the processing system 1020 may execute the robotic control SW module 1032 to control the robotic arm to manipulate the medical instrument based on the received user inputs. The processing system 1320 also may execute the graphical interface SW module 1034 to display a graphical interface including an image depicting the medical instrument in a field-of-view (FOV) of the camera and a visual indicator indicating a mapping of the plurality of directions to the medical instrument in the FOV of the camera. In some implementations, the visual indicator may indicate that one or more visual features disposed on the elongate shaft are associated with one or more directions, respectively, of the plurality of directions (such as described with reference to FIG. 8). In some other implementations, the visual indicator may be overlaid on the medical instrument in the image (such as described with reference to FIGS. 9A and 9B). In such implementations, the processing system 1020 may further determine the mapping of the plurality of directions to the medical instrument based on the pose of the medical instrument and the pose of the camera.

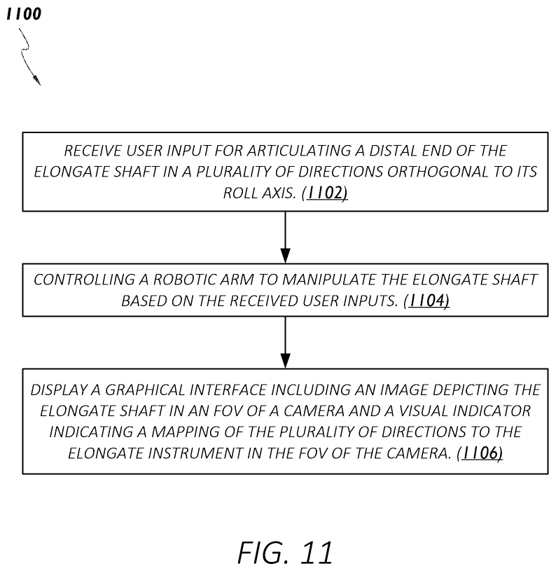

FIG. 11 shows an illustrative flowchart depicting an example operation 1100 for controlling a medical instrument having an elongate shaft, according to some implementations. In some implementations, the example operation 1100 may be performed by a medical system such as the medical system 100 of FIG. 1 or the system 1000 of FIG. 10.

The medical system receives user input for articulating a distal end of the elongate shaft in a plurality of directions orthogonal to its roll axis (1102). The medical system further controls a robotic arm to manipulate the elongate shaft based on the received user inputs (1104). The medical system also displays a graphical interface including an image depicting the elongate shaft in an FOV of a camera and a visual indicator indicating a mapping of the plurality of directions to the elongate shaft in the FOV of the camera (1106). In some aspects, the visual indicator may indicate that a visual feature disposed on the elongate shaft is associated with a respective direction of the plurality of directions.

In some other aspects, the medical system may further receiving first sensor data indicating a pose of the medical instrument, receiving second sensor data indicating a pose of the camera, and determining the mapping of the plurality of directions to the medical instrument based on the pose of the medical instrument and the pose of the camera. In some implementations, the visual indicator may be overlaid on the medical instrument in the image. In some implementations, the controlling of the robotic arm may include locking the plurality of directions to a coordinate frame associated with the camera.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A medical instrument, comprising:
an elongate shaft having a distal end configured to articulate in a plurality of directions orthogonal to its roll axis;
a first visual feature disposed on a first portion of the elongate shaft associated with a first direction of the plurality of directions; and
a second visual feature disposed on a second portion of the elongate shaft different than the first portion so that the elongate shaft is visually asymmetric about the roll axis.

2. The medical instrument of claim 1, wherein the first and second visual features are disposed on an outer surface of the elongate shaft.

3. The medical instrument of claim 1, wherein the first direction points radially to the first portion of the elongate shaft.

4. The medical instrument of claim 1, wherein the second portion of the elongate shaft is associated with a second direction of the plurality of directions opposite the first direction.

5. The medical instrument of claim 1, wherein the first and second visual features comprise different colors.

6. The medical instrument of claim 1, wherein the first and second visual features comprise different patterns.

7. The medical instrument of claim 1, wherein the first visual feature comprises a plurality of markings that are spaced apart by a predetermined distance.

8. The medical instrument of claim 1, further comprising:
a third visual feature disposed on a third portion of the elongate shaft associated with a third direction of the plurality of directions orthogonal to the first direction.

9. A system comprising:
a medical instrument having an elongate shaft;
a robotic arm configured to manipulate the medical instrument; and
control circuitry configured to:
receive user input for articulating a distal end of the elongate shaft in a plurality of directions orthogonal to its roll axis;
control the robotic arm to manipulate the medical instrument based on the received user inputs; and
display a graphical interface including an image depicting the medical instrument in a field-of-view (FOV) of a camera and a visual indicator indicating a mapping of the plurality of directions to the medical instrument in the FOV of the camera.

10. The system of claim 9, wherein the medical instrument comprises:
a first visual feature disposed on a first portion of the elongate shaft, the visual indicator indicating that the first visual feature is associated with a first direction of the plurality of directions.

11. The system of claim 10, wherein the medical instrument further comprises:
a second visual feature disposed on a second portion of the elongate shaft different than the first portion, the visual indicator further indicating that the second visual feature is associated with a second direction of the plurality of directions opposite the first direction.

12. The system of claim 11, wherein the medical instrument further comprises:
a third visual feature disposed on a third portion of the elongate shaft, the visual indicator further indicating that the third visual feature is associated with a third direction of the plurality of directions orthogonal to the first direction and the second direction.

13. The system of claim 9, wherein the control circuitry is further configured to:
receive first sensor data indicating a pose of the medical instrument;
receive second sensor data indicating a pose of the camera; and
determine the mapping of the plurality of directions to the medical instrument based on the pose of the medical instrument and the pose of the camera.

14. The system of claim 13, wherein the visual indicator is overlaid on the medical instrument in the image.

15. The system of claim 13, wherein the control of the robotic arm locks the plurality of directions to a coordinate frame associated with the camera.

16. A method for controlling a medical instrument having an elongate shaft, comprising:
receiving user input for articulating a distal end of the elongate shaft in a plurality of directions orthogonal to its roll axis;
controlling a robotic arm to manipulate the elongate shaft based on the received user inputs; and
displaying a graphical interface including an image depicting the elongate shaft in a field-of-view (FOV) of a camera and a visual indicator indicating a mapping of the plurality of directions to the elongate shaft in the FOV of the camera.

17. The method of claim 16, wherein visual indicator indicates that a visual feature disposed on the elongate shaft is associated with a respective direction of the plurality of directions.

18. The method of claim 16, further comprising:
receiving first sensor data indicating a pose of the medical instrument;
receiving second sensor data indicating a pose of the camera; and
determining the mapping of the plurality of directions to the medical instrument based on the pose of the medical instrument and the pose of the camera.

19. The method of claim 18, wherein the visual indicator is overlaid on the medical instrument in the image.

20. The method of claim 18, wherein the controlling of the robotic arm comprises locking the plurality of directions to a coordinate frame associated with the camera.
